(19) United States
(12) Patent Application Publication
(10) Pub. No.: US 2025/0000480 A1
(43) Pub. Date: Jan. 2, 2025

(54) WELLNESS AND HEALTHCARE MONITORING DEVICE

(71) Applicant: Quadralynx, Inc., Spokane, WA (US)

(72) Inventors: Mark VanDam, Spokane, WA (US); Gavin La Rowe, Spokane, WA (US)

(21) Appl. No.: 18/756,857

(22) Filed: Jun. 27, 2024

Related U.S. Application Data
(60) Provisional application No. 63/524,667, filed on Jul. 1, 2023.

(51) Int. Cl. A61B 7/04; A61B 5/00; A61B 5/16

(52) U.S. Cl. CPC A61B 7/04; A61B 5/165; A61B 5/7246; A61B 5/7264

(57) ABSTRACT

A device is wearable by a user and includes one or more microphones, an output component, and a network interface. The device receives, from the one or more microphones, audio data associated with sound captured within an environment of the user, and determines that the sound is associated with the user. Based on the sound being associated with the user, the device determines one or more biomarkers associated with the sound. A threshold associated with the user having a pathological or behavioral state is determined and when the one or more biomarkers satisfy the threshold, the sound is determined to be indicative of the user having the pathological or behavioral state. The device outputs a first notification on the output component and sends data associated with a second notification to be output on an electronic device associated with a caregiver of the user.

### CROSS-REFERENCE TO RELATED APPLICATIONS

This application claims priority to U.S. Provisional Patent Application No. 63/524,667, filed Jul. 1, 2023, entitled “Wearable Smart-Sensor and Biofeedback Devices and Methods for Detecting, Analyzing and Reporting Disease, Health Concerns and Other Adverse Welfare Conditions in Human Subjects,” the entirety of which is herein incorporated by reference.

### BACKGROUND

Monitoring wellness, human health, and welfare is often labor-intensive and time-consuming. Despite modern tools and advancements, however, diagnosing, treating, and managing human welfare remains insufficient. For example, conventional monitoring and treatment often focuses on costly and time-consuming in-person visits. While devices and/or wearables exist, these devices have limited functionality and inconclusive results. As such, current methods and tools for effective human health monitoring are outdated, ineffective, and limited.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features. The systems depicted in the accompanying figures are not to scale and components within the figures may be depicted not to scale with each other.

FIG. 1 illustrates an example healthcare monitoring device used to determine a welfare, behavioral, and/or pathological state of a user, according to examples of the present disclosure.

FIG. 2 illustrates a plurality of healthcare monitoring devices used to determine a welfare, behavioral, and/or pathological state of a user, according to examples of the present disclosure.

FIG. 3 illustrates an example process in which the wellness and healthcare monitoring device determines a welfare, behavioral, and/or pathological state of a user, according to examples of the present disclosure.

### DETAILED DESCRIPTION

This application is directed, at least in part, to monitoring user welfare to determine one or more behavioral and/or pathological states of the user, according to examples of the present disclosure. In an embodiment, sensor data generated from one or more sensors may be used to determine user welfare for detecting behavioral and/or pathological states, such as diseases, distresses, accidents, injuries, etc., to provide care, treatment, and/or management. In an embodiment, any number of sensors may be used to acoustically and non-acoustically monitor the user. For example, the sensors may capture data indicative of sounds generated by the user, movements of the user, vitals of the user, behavior of the wearer, etc. The sensor data may be analyzed to determine biomarkers associated with the user, where the biomarkers are used to determine a welfare of the user for deducing behavioral and/or pathological states of the user. In an embodiment, the biomarkers may be compared to one or more reference biomarkers to deduce the behavioral and/or pathological states. The user welfare and/or pathological states may be determined in real-time and/or over a period of time (e.g., day, month, year, etc.). In an embodiment, based at least in part on the welfare, behavioral, and/or pathological state, a caregiver may be notified to provide care, treatment, etc.

In an embodiment, the techniques described herein may be implemented within, or at least in part, by a device. In an embodiment, the device may be considered a wellness and healthcare monitoring device. For example, as will be discussed herein, the wellness and healthcare monitoring device may, in an embodiment, generate or utilize sensor data associated with acoustics of the user, non-acoustics of the user, and/or an environment of the user as a way to monitor the user and/or provide feedback to the user, a caregiver (e.g., physician, therapist, etc.) of the user, and so forth. However, although described as a wellness and healthcare monitoring device, the systems, methods, and techniques described herein may be utilized within applications other than healthcare.

In an embodiment, the wellness and healthcare monitoring device may be a compact device worn by the user. The wellness and healthcare monitoring device may be worn in any manner by the user, such as in the form of bracelets, watches, stick-on arrays, eyeglasses, caps, hearing aids, brooches, tics, lanyards, earrings, etc. The wellness and healthcare monitoring device may include suitable attachment mechanisms, such as hook and loop, clasps, magnets, cords, adhesives, chains, etc., for coupling (e.g., pinning, hanging, etc.) in any manner to the user. However, although described herein as a device worn by the user, in an embodiment, the techniques described herein may not be limited to such devices. For example, a device (e.g., stand-alone device, integrated device, etc.) employing the methods described herein may be placed on a shelf, counter, wall, etc., within an environment where the user resides or occupies.

The wellness and healthcare monitoring device may include a housing in which components of the wellness and healthcare monitoring device are disposed. The housing may include any shape, such as cylindrical, square-shaped, etc. The housing may be manufactured from suitable materials, such as plastics, composites, metal, etc., and using any manufacturing process(es), such as injection molding, stamping, blow molding, etc. The housing may also be contaminate-resistant to prevent the ingress of liquid, dust, or contaminants into the housing. Additionally, the wellness and healthcare monitoring device may include suitable processing and internal components to detect and manage welfare, behavioral, and/or pathological states of the user. For example, as will be discussed herein, the wellness and healthcare monitoring device may include sensor(s), microphone(s), battery(ies), processing components (e.g., processors, memory, system on a chip (SoC), or integrated circuits (IC), etc.), etc. disposed within the housing. In an embodiment, the wellness and healthcare monitoring device may include I/O components, such as buttons, switches, lighting elements, and/or a display for outputting information associated with the user. The display may include an organic light emitting diode (O/LED), (in-plane switching (IPS) or thin-film-transistor (TFT)) liquid crystal display (LCD), laser video display (LVD), vibrotactile, haptic, or other applications.

As introduced above, the wellness and healthcare monitoring device may diagnose, manage, and/or monitor the welfare, behavioral, and/or pathological state or changes of the user. In an embodiment, the wellness and healthcare monitoring device may be administered to the user to diagnose, manage, and/or monitor the welfare, behavioral, and/or pathological state or changes in state. While the cause of certain welfare, behavioral, and/or pathological states may be unknown, characteristic(s) associated with the welfare, behavioral, and/or pathological states are often known. The wellness and healthcare monitoring device includes the sensor(s) to determine the characteristic(s) of the user, which, in turn, are compared to reference characteristics for determining the welfare, behavioral, and/or pathological states.

The wellness and healthcare monitoring device may include any number of sensor(s) to monitor the user and/or the environment of the user for determining the welfare, behavioral, and/or pathological states. Example sensor(s) include but are not limited to microphone(s), piezoelectric sensor(s), accelerometer(s), camera(s) or optical sensors, temperature sensor(s), heart rate sensor(s), humidity sensor(s), inertial measurement unit (IMU) sensor(s), gyroscope, ECC sensor(s), blood glucose sensor(s), breathing sensor(s), geo- and spatial location sensors such as GPS or RFID sensor(s), blood oxygen sensor(s), Hall sensor(s), electrodermal sensors such as galvanic skin response, passive infrared (PIR) sensor(s), etc. In an embodiment, the sensor(s) may be used to identify vitals associated with the user, activities of the user, and non-activities of the user, as well as to characterize the vitals, activities, and/or non-activity. For example, the sensor(s) may be used to determine active or volitional activities of the user (e.g., vocalizations, movement, eating, sleeping, etc.) and/or non-activates, passive vegetative, automatic, autonomic (sympathetic, parasympathetic, enteric)/involuntary activities/states of the user (e.g., non-vocalizations, breathing, heart rate, blood oxygen level, etc.). Moreover, the sensor(s) may be used to determine any number of sounds or signals generated by or emanating from the user, whether audio, vocals, speech, murmuring, mumbling, etc.

The sensor(s) may capture audio data and/or sensor data that is analyzed to determine the characteristic(s) of the user. In an embodiment, the characteristic(s) may be associated with the sounds, behavior, vitals, activities, and/or non-activities of the user. In an embodiment, the characteristic(s) may be used to determine biomarker(s) associated with vocalizations and/or sounds of the user. For example, the biomarker(s) may be associated with pitch, intonation, tone, pauses, phonation, and/or changes associated with speech of the user. The biomarker(s) may also include features such as articulation, decreased energy in the higher parts of a harmonic spectrum, imprecise articulation of vowels and consonants, fundamental frequency, voicing, windowed and absolute syllable/sonorant-peak rates, SNR, temporal and spectral voice characteristics, frequency, spectral/cepstral cues, vocal quality/stability (e.g., shimmer, jitter, harmonic to noise ratio), prosody, temporal output, amplitude stability, etc. Biomarker(s) may also be associated with non-acoustic characteristic(s) of the user, such as motor processes, vitals, EEG, EKG, motion, fatigue, physical activity, posture, gait, location, as well as environment biomarkers such as temperature, air quality, location, date/time, etc.

The wellness and healthcare monitoring device may include analog and digital converters that assist in capturing, processing, and storing signals collected from the microphone(s) and sensor(s), respectively. The signals from the microphone(s) and sensor(s) may be stored in a dataset including raw and/or normalized or transformed values. The audio signals or sensor signals may be processed as data, which is analyzed to determine the biomarkers.

In an embodiment, the microphone(s) may include at least two microphones spaced apart from one another within the housing. For example, the housing may include a first end and a second end spaced apart from the first end. A first of the microphones may be located closer to the first end than the second end, while a second of the microphones may be located closer to the second end than the first end. Any number of microphones, however, may be included within the wellness and healthcare monitoring device. In an embodiment, the microphone(s) may be used to determine a directionality (e.g., time of flight, velocimetry, time-delay analytics, etc.) of the signal and/or whether the signal corresponds to the sounds of the user wearing the wellness and healthcare monitoring device. For example, the microphone(s) may receive respective audio signals that represent sounds within the environment. When worn by the user, the microphones may be spaced apart by different distances from sound sources of the user (e.g., mouth of the user, stomach, etc.). As such, the microphone(s) are spaced apart by a different distance from the sound sources of the user to discern sounds generated by the user from other sources of sound within the environment.

The audio signals captured by the microphone(s) may be cross-correlated with one another. For example, cross-correlation may applied between audio signals detected by the microphones to determine a time difference of arrival (TDOA) for the correlated signals. In other words, the differently spaced microphones from the mouth of the user have different time of arrival (TOA) values. This allows the calculation of an angle from which the detected signals originate relative to a line intersecting the two microphones.

In an embodiment, the wellness and healthcare monitoring device may be configured to identify sounds emanating or generated from the user, as compared to sounds generated from other sources within an environment of the user. As introduced above, the sound of the user may include vocalizations (e.g., speech, moaning, grunting, etc.) generated from a mount of the user, but sound generated by digestion, body parts, etc. of the user. Moreover, the microphone(s) may capture sound or other acoustics of an environment of the user (e.g., weather, sounds of other people, etc.). In an embodiment, sounds may also be associated with the user, as compared to other sources of sound, based on detecting vocal vibrations through changes in pressure, breathing, acceleration, temperature, strain, force, etc. The wellness and healthcare monitoring device may detect vocal pathological or distress biomarkers, such as sonic or other detectable human vocal biomarkers associated with a targeted disease condition or welfare disorder. In an embodiment, the wellness and healthcare monitoring device detects one or more sonic markers associated with disease or distress, characterized by a diagnostic vocal, non-vocal acoustic or non-acoustic detected signal.

Capturing sounds of the user allows for the determination of whether the user includes a behavioral and/or pathological state that warrants care, intervention, treatment, etc. As such, sounds (e.g., pathological sounds) emitted from the user may be detected and analyzed to identify and inform a caregiver regarding the pathological state of the user, whether normal, suspect, or determined pathological or distress experienced by the user. In an embodiment, these different vocalizations may have or be characterized by reference signatures. As will be discussed herein, in an embodiment, signatures (e.g., audio, motion, etc.) may be compared against reference signatures to determine the welfare, behavioral, and/or pathological state of the user.

In addition to acoustics, accelerometers may capture sensor signals, or sensor data, to determine behaviors of the users, such as movements (e.g., running, digestion, etc.), vitals (e.g., temperature, pulse rate, respiration rate, blood pressure, etc.), activities (e.g., breathing, heart rate, congestion, coughing, etc.), and so forth. For example, heart rate sensor(s) may be used to determine health of the user (e.g., electrocardiogram, photoelectric pulse wave, blood pressure measurement, phonocardiography, etc.), weather sensor(s) may be used to determine weather conditions (e.g., temperature of the user and/or environment), and so forth.

The biomarkers may be determined from audio data generated by microphone(s) of the wellness and healthcare monitoring device, and/or sensor data generated from sensor(s) of the wellness and healthcare monitoring device. The biomarkers may be compared against reference biomarkers or thresholds, for example, to determine the welfare, behavioral, and/or pathological state of the user. In an embodiment, the behavioral and/or pathological state of the user may be determined based at least in part on the welfare. In an embodiment, the reference biomarkers may be considered diagnostic or classificatory biomarkers in that the biomarkers are compared to the reference biomarkers to determine the welfare, behavioral, and/or pathological state or changes in state of the user. For example, the wellness and healthcare monitoring device may detect one or more biomarkers associated with congenital heart disease, which may be characterized by heart murmurs, rapid breathing, shortness of breath, rapid heartbeat, etc. (e.g., congenital heart disease biomarkers). Here, the wellness and healthcare monitoring device may determine characteristic(s) of the user for determining the biomarkers, and in turn, may compare the biomarkers singly or in combination/interaction to reference biomarkers indicative of congenital heart disease. Based on this comparison, the wellness and healthcare monitoring device may determine whether the characteristic(s) of the user are indicative of congenital heart disease.

The wellness and healthcare monitoring device may be useful to detect any number of types of pathological conditions, distress states, behavior, and a wide range of other activities and circumstances detectable from a user and/or their environment. In an embodiment, the wellness and healthcare monitoring device may be configured to detect the particular welfare, behavioral, and/or pathological state of the user. In this sense, the wellness and healthcare monitoring device may be tailored to the user (e.g., with a known disease, for example) or may be used to diagnose welfare, behavioral, and/or pathological states. Depending upon the welfare(s), behavioral, and/or pathological state(s), comparable sets of reference biomarkers may be employed.

For example, the wellness and healthcare monitoring device may be specifically programmed and/or optimized for the specific biomarkers and/or characteristic(s) that are already established or known for a behavioral and/or pathological state for a given disease, as well as the disease, the user, and/or the environments. For example, continuing with the above example, if a user has congenital heart disease, the wellness and healthcare monitoring device may be programmed to monitor for characteristics (e.g., heart rate, breathing patterns, movement, etc.) indicative of congenital heart disease, as compared to other diseases. Moreover, in an embodiment, the wellness and healthcare monitoring device (or the sensors) may be calibrated to each disease, behavioral and/or pathological state of the disease, the user, the environment, location, etc. The comparison between the biomarkers and the reference biomarkers or thresholds may determine a statistical likelihood of the welfare, behavioral, and/or pathological state of the user.

As introduced above, reference biomarker(s), whether acoustic or non-acoustic, may be captured, stored as sample data, and analyzed in conjunction with audio data and/or sensor data detected by the wellness and healthcare monitoring device to detect, diagnose, and manage a wide range of welfares, behavioral, and/or pathological states. Example diseases include, but are not limited to, infectious diseases, as well as metabolic disorders, gate disorders, respiratory diseases, gastrointestinal diseases, a wide range of non-pathological behaviors and activities (both volitional and autonomic), etc. Early detection and monitoring of these diseases and other adverse welfare states greatly enhance human health and welfare and may minimize the need for costly medical intervention. In an embodiment, the welfare of the user may be associated with healthy, sick, diseased, etc. In an embodiment, the welfare of the user may be used to classify or determine the behavioral and/or pathological states of the user. Pathological states may be related to, but are not limited to, respiration, feeding, water intake, movement, digestive, gastrointestinal, disease, metabolism, gate, and so forth. These pathological states may be correspondingly utilized to identify, characterize, diagnose, treat, etc. diseases (e.g., states, factors, etc.), disabilities, distresses, welfare disturbances, pathologies, activities, congenital defects, effects of medications, environmental effects, accidents, harmful physical events, etc. associated with the user. Sensor and sampled data may also be used to identify and classify non-pathological states that are also of interest to behaviors, activities, states, changes, and observations of the device user and those interacting with the device user. Biomarkers of non-pathological behavior and states are also of interest and can be the target of classification and “diagnostic” determinations that contribute to interventions, suggestions, analyses, and so on for bearing on quality-of-life, improved performance, reflection, monitoring, aptitude, assessment, tracking of behavior/conditions, and other data.

In an embodiment, the wellness and healthcare monitoring device may generate fingerprint(s), unique signature(s), or phenotype(s) based at least in part on the biomarkers. The fingerprint(s) may be individual to the user and permit the user to be distinguished from one another. In an embodiment, the use of the fingerprint(s) may discern sound generated from the user from other sounds emanating within the environment. This allows the sound to be associated with a certain user to accurately determine the welfare, behavioral, and/or pathological state of a particular user. The fingerprint(s) may also improve the accuracy of determining welfare, behavioral, and/or pathological states. For example, in an embodiment, the biomarkers may be used to generate fingerprint(s) associated with the user, and the fingerprint(s) may be analyzed to determine whether the fingerprint(s) is associated with behavioral and/or pathological states. In an embodiment, the fingerprint(s) may characterize the sounds, activities, movements, vitals, and the fingerprint(s), for example, and may be compared to one or more reference fingerprint(s) to determine whether the welfare, behavioral, and/or pathological states based on a comparison to reference fingerprint(s).

The fingerprint(s) may be based at least in part on the audio data and/or the sensor data. In doing so, acoustics and non-acoustic characteristic(s) may be used to indicate a certain welfare, behavioral, and/or pathological state of the user. For example, fingerprint(s) may associate acoustic sounds (e.g., breathing) with non-acoustic sounds (e.g., an acceleration of the chest during breathing) for comparison to characteristic(s) of the user to determine the welfare, behavioral, and/or pathological states. In other words, audio data and/or sensor data may be used to characterize the user, thereby generating a fingerprint associated with the sounds, non-sounds, vitals, movement, etc. of the user, and such fingerprint may be compared to reference fingerprints to determine the welfare, behavioral, and/or pathological states. Through this comparison, like characteristics (e.g., sound to sound, movement to movement, etc.) may be compared with one another to determine the welfare, behavioral, and/or pathological states.

The wellness and healthcare monitoring device may utilize other data associated with the user to determine the welfare, behavioral, and/or pathological state of the user. For example, a profile of the user may store information such as age, sex, medications, past medical history, location, etc. This information may be used to increase accuracy in detecting the welfare, behavioral, and/or pathological state of the user. For example, the information may be considered when determining the welfare, behavioral, and/or pathological state of the user. That is, if a user is taking medications and is prone to certain diseases based on prior diagnoses, the wellness and healthcare monitoring device may utilize such information when determining the welfare, behavioral, and/or pathological states.

The wellness and healthcare monitoring device may output the notification(s) in an attempt to notify the user of their welfare, behavioral, and/or pathological state. The notification(s) may also be sent to other devices. For example, a caregiver of the user may receive the notification(s) as a way to be notified about welfare and/or pathological states, diagnose the welfare and/or pathological states, treat the welfare and/or pathological states, and/or manage the welfare and/or pathological states.

The notification(s) may be output in real-time, or substantially real-time, to provide near-instantaneous feedback to the user regarding their welfare, behavioral, and/or pathological state. In an embodiment, the notification(s) may be audible, visual, olfactory, electronic, haptic, etc., and/or any combination thereof. For example, the wellness and healthcare monitoring device may include a speaker that outputs audio (e.g., deeps, tones, instructions, etc.), lighting elements that illuminate (e.g., patterns, colors, etc.), and/or a motor that vibrates. Noted above, the notification(s) may also be output on a display of the wellness and healthcare monitoring device. The wellness and healthcare monitoring device may output the notification(s) for a predetermined amount of time, until the user responds, etc. In an embodiment, the wellness and healthcare monitoring device may tailor the notification(s) based on one or more preference(s) stored in association with a profile of the user. For example, the profile may indicate a type of notification(s) to output, as well as their associated intensity (e.g., volume, luminosity, etc.), duration, type (e.g., sound-haptic-sound), patterns (e.g., low haptic-medium haptic-low haptic, etc.), combinations thereof, etc. The wellness and healthcare monitoring device may access the preference(s) as stored in the profile when outputting the notification(s).

In addition to real-time feedback as described above, asynchronous reports may also be delivered to the user and stored in the software. For example, a daily histography digest of performance and behavior may be delivered via email or software (e.g., a mobile application) to the user. Performance data may also be delivered to a database collection performance characteristics from many users constituting the basis for additional software and algorithm changes. The application and/or software may be embedded into the wellness and healthcare monitoring device or may be external software capable of being instantiated into external hardware such as a mobile phone, tablet, or computer. Although the techniques are described herein as being performed by the wellness and healthcare monitoring device, the techniques may be embodied as software or an application operating on a device (e.g., mobile phone). Suitable APIs may interact and/or utilize sensor(s) of device(s), such as camera(s), microphone(s), location, temperature, etc. to carry out the operations described herein.

In an embodiment, the wellness and healthcare monitoring device may utilize artificial intelligence (AI) and machine learning (ML) models to improve the accuracy of determining the welfare, behavioral, and/or pathological states of the user. To clarify, AI and ML techniques, which are described and detailed with examples below, are used as encompassing cover terms for “smart” and algorithmic collection, processing, analyzing, databasing, managing, summarizing, and using data in an automated and large-scale fashion. The ML model(s) may be trained from a database (e.g., historical data, such as audio data that includes respiration, feeding, water intake, movement, digestive, gastrointestinal, disease, metabolism, gate, vitals, disabilities, distresses, welfare disturbances, pathologies, activities, congenital defects, effects of medications, environmental effects, accidents, harmful physical events, etc. of the user) to analyze the data captured by the sensor(s) and microphone(s) for identifying the biomarkers and determining the welfare, behavioral, and/or pathological states. The ML model(s) may determine the biomarkers and assess the biomarkers in comparison to information stored in the database (e.g., reference characteristics, reference fingerprint(s), etc.) to determine the welfare, behavioral, and/or pathological states.

As part of the ML model(s) analyzing the audio data and/or the sensor data, the ML model(s) may label the characteristic(s) of the activities, non-activities, and/or behavior of the user singly or in combination, for example, the biomarkers, to indicate whether the characteristic(s) are associated certain welfare, behavioral, and/or pathological states. In an embodiment, an output of the ML model(s) may indicate whether the user has a particular welfare, behavioral, and/or pathological state (e.g., healthy, diseased, injured, etc.). In an embodiment, the ML model(s) may determine the characteristic(s) for comparison to respective reference characteristics to determine whether activities, non-activities, and/or behaviors of the user are indicative of, or are not indicative of, a particular welfare, behavioral, and/or pathological state. The ML model(s) may use any number of characteristic(s) for determining the welfare, behavioral, and/or pathological states.

In an embodiment, the ML model(s) may generate scores, metrics, classifications, or determinations that are associated with the characteristic(s) of the welfare, behavioral, and/or pathological states. For example, the ML model(s) may generate scores associated with the biomarkers, welfare, behavioral, and/or pathological states. For example, if the scores are greater than a predetermined threshold (which varies depending on the particular data and desired information relayed in return), the ML model(s) may return a quantitative confidence that the characteristics are associated with a particular welfare, behavioral, and/or pathological states. In an embodiment, the ML model(s) may compare fingerprint(s)/phenotype(s) of the user, as generated from the audio data and/or sensor data, against one or more reference fingerprints. A behavioral and/or pathological state may be assessed with regard to the model parameters, and a discrete or probabilistic classification will be generated. The classification may be stored, processed, and used for feedback (as needed).

In an embodiment, the ML model(s) may receive or be trained from characteristic(s) that are specific to any number of diseases. The ML model(s), having been trained, may receive the audio data and/or sensor data to predict and/or identify the behavioral and/or pathological states of the user. In an embodiment, the ML model(s) may be compared characteristic(s) of the audio data and/or the sensor data with reference characteristic(s) associated with the diseases, respectively. As an example, in the context of opioid use, the ML model(s) may identify correlations (e.g., factors, variables, features, etc.) to predict and/or determine whether a user has an opioid addiction. Such correlations may be based on a given condition or state associated with a disease, disorder, condition, socioeconomic status, location, etc.

The ML model(s) may be trained to determine characteristic(s) of the audio data and/or sensor data, or characteristic(s) of an individual behavioral and/or pathological state. The ML model(s) may be trained from data collected from users with similar demographics (e.g., sex, age, ethnicity, etc.). For example, the ML model(s) may be trained from amplitudes associated with the audio data, sex, race, age, disease state, audiological status, medical history, socioeconomic status (SES), geographical location, etc., as well as environmental factors (e.g., time of day, medication schedule, auditory and acoustic environment, etc.). The ML model(s) may be dynamic and retrained to improve an accuracy in determining welfare, behavioral, and/or pathological states.

Although the wellness and healthcare monitoring device is described as processing, analyzing, and/or determining the characteristic(s) of the user, the biomarkers, welfare, behavioral, and/or pathological states, etc., in an embodiment, the wellness and healthcare monitoring device may communicatively couple to one or more computing devices, resource(s), etc. which may additionally or alternatively process, analyze, and/or determine the characteristic(s) of the user, the biomarkers, and welfare, behavioral, and/or pathological states. For example, the wellness and healthcare monitoring device may receive or capture sensor data that is further transmitted to remote computing resource(s) for analysis. The remote computing resource(s) may receive the sensor data from any number of the wellness and healthcare monitoring device(s) or separate device(s). In an embodiment, the remote computing resource(s) may analyze the sensor data, among other data, to determine the biomarkers, welfare, pathological states, etc. Thereafter, the remote computing resource(s) may transmit notification(s) to the wellness and healthcare monitoring device, the device of the caregiver, healthcare provider, other organization, and so forth. Any level of split processing may be performed by the wellness and healthcare monitoring device, the device of the caregiver, the remote computing resource(s), and/or other devices, systems, networks, etc.

In an embodiment, the wellness and healthcare monitoring devices may be used in conjunction with one another, such as in a system, to determine the user welfare, behavior, and/or pathological states or activity. For example, a first wellness and healthcare monitoring device (i.e., a first system component) worn on the user may be used in conjunction with a second wellness and healthcare monitoring device (i.e., a second system component) placed within an environment of the user. In such instances, the wellness and healthcare monitoring device may be the same or different, for example, such as in size, functionality, capabilities, etc. For example, the first wellness and healthcare monitoring device may include first sensors that are different from the second sensors of the second wellness and healthcare monitoring device. In instances where a plurality of the wellness and healthcare monitoring devices are used, the wellness and healthcare monitoring devices may be communicatively coupled to one another via one or more network interface(s) (e.g., Bluetooth, Cellular, etc.). Any combination of the wellness and healthcare monitoring devices worn on the user and/or off the user may be used.

The wellness and healthcare monitoring device may be used within a plurality of environments and/or for any suitable purposes. For example, the wellness and healthcare monitoring device may find use within healthcare, neurology, speech pathology, speech training, physical therapy, occupational therapy, professional caregiving, physical therapy, occupational therapy, occupational safety, sporting events, home environment, leisure activities, during periods of rest or activity, social gatherings/events, during meal preparation, personal hygiene care, sleeping, reading, etc. The wellness and healthcare monitor device may be utilized to determine any disease, such as Parkinson's disease, Alzheimer's disease, Frontotemporal dementia, Vascular dementia, ALS, stroke, hereditary Prior disease, etc. The wellness and healthcare monitoring device may be utilized to determine prodromal, symptomatic, unknown, suspected, or other markers and biomarkers of disease and disorder. The wellness and healthcare monitoring device may be utilized to determine and assist in non-disease related behavior and states, including classifications and descriptions of states, activities, and behaviors that contribute to wellness, well-being, quality of life, mobility, active lifestyles, independent living, mood, psychiatric/psychological/psychosocial well-being, and so on.

The present disclosure provides an overall understanding of the principles of the structure, function, device, and system disclosed herein. One or more examples of the present disclosure are illustrated in the accompanying drawings. Those of ordinary skill in the art will understand and appreciate that the devices, the systems, and/or the methods specifically described herein and illustrated in the accompanying drawings are non-limiting embodiments. The features illustrated or described in connection with one embodiment, or instance, may be combined with the features of other embodiments or instances. Such modifications and variations are intended to be included within the scope of the disclosure and appended claims.

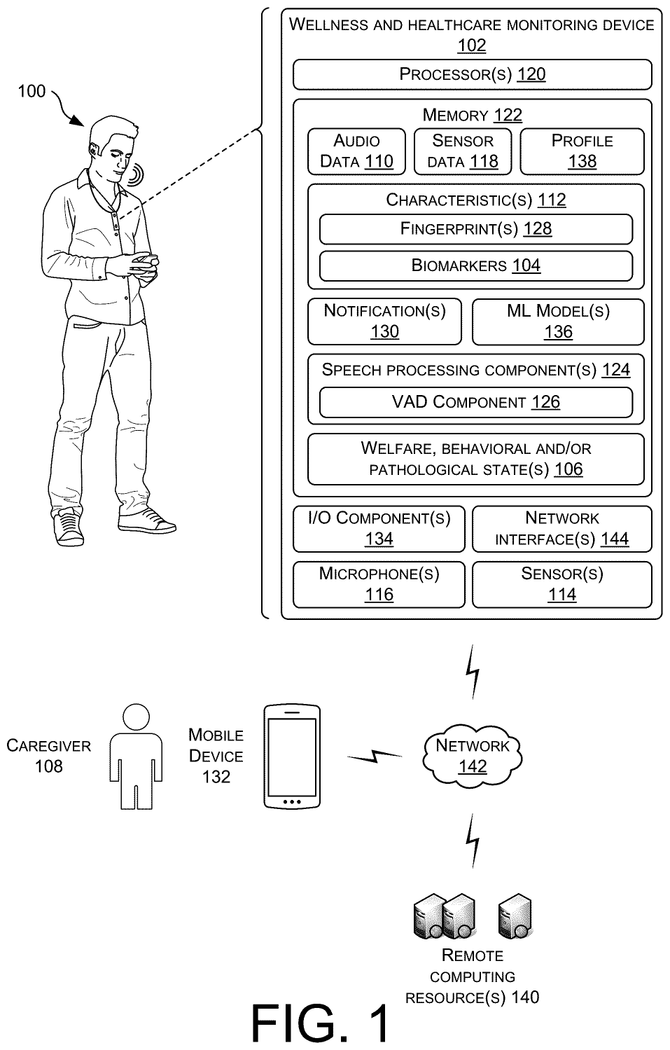

FIG. 1 illustrates an example user 100 wearing a wellness and healthcare monitoring device 102, according to examples of the present disclosure. Details of the wellness and healthcare monitoring device 102 are discussed herein, however, generally, the wellness and healthcare monitoring device 102 may determine biomarkers 104 of the user 100 for detecting welfare, behavior, and/or pathological states 106 of the user 100. Detection of the welfare, behavior, and/or pathological states 106 allows for a caregiver 108 (e.g., physician, therapist, etc.) to treat and/or manage the user 100. In an embodiment, the wellness and healthcare monitoring device 102 may be similar to the devices and/or include similar functionality as the device discussed in U.S. patent application Ser. No. 18/755,471, filed Jun. 26, 2024, the entirety of which is herein incorporated by reference.

The wellness and healthcare monitoring device 102 may be a compact, wearable, or peripheral device. In an embodiment, the wellness and healthcare monitoring device 102 may be worn on a body of the user 100. In an embodiment, the wellness and healthcare monitoring device 102 may be small, non-invasive, non-intrusive, lightweight, and discreet in nature. As shown in FIG. 1, the wellness and healthcare monitoring device 102 may be worn as a necklace or lanyard. However, the wellness and healthcare monitoring device 102 may be attached or worn by the user 100 using mechanisms such as hook and loop, clasps, magnets, cords, chains, clips, belts and buckles, cords, pins, adhesives, etc. for coupling (e.g., pinning, hanging, etc.) in any manner to the user 100. Still, in an embodiment, the wellness and healthcare monitoring device 102 may be sub-dermally implanted, injected, or ingested. As such, although shown in a particular embodiment in FIG. 1, the wellness and healthcare monitoring device 102 may be attached or worn by the user 100 in other manners.

In an embodiment, the wellness and healthcare monitoring device 102 may be separate from the user 100, such as not being worn by the user 100, placed in proximity to the user 100 or caregiver 108. For example, the wellness and healthcare monitoring device 102 may be positioned relative to the user, such as on a shelf, stand, etc. to monitor the user 100 and/or an environment of the user 100.

The wellness and healthcare monitoring device 102 may capture, generate, or receive audio data 110, such as vocalizations, sound, noises, etc. of the user 100 or the environment and process the audio data 110 to detect the welfare, behavior, and/or pathological states 106. Based on processing the audio data 110, the wellness and healthcare monitoring device 102 may provide real-time feedback to the caregiver 108 that prompts the caregiver 108 to treat or manage the user 100, for example, based at least in part on the welfare, behavior, and/or pathological states 106. For example, the caregiver 108 may provide assistance to the user 100, administer vaccines, medications, etc. In an embodiment, the wellness and healthcare monitoring device 102 may continuously capture and process the audio data 110 to provide real-time feedback and/or triggered to capture the audio data 110.

In an embodiment, the wellness and healthcare monitoring device 102 may process the audio data 110 to determine characteristic(s) 112 of the vocalizations of the user 100. The characteristic(s) 112 may be compared to reference characteristics, thresholds, etc. for detecting when the vocalizations indicate certain welfare, behavior, and/or pathological states 106 or include characteristic(s) associated with certain welfare, behavior, and/or pathological states 106. In an embodiment, the characteristic(s) 112 may include or be represented by biomarkers 104, where the biomarkers 104 may identify or be associated with the characteristic(s) 112. As an example, the wellness and healthcare monitoring device 102 may detect one or more of the biomarkers associated with congenital heart disease, diabetes, measles, diphtheria, etc. For example, the wellness and healthcare monitoring device 102 may detect a simultaneous combination of increased electrodermal activity and decreased heart rate in a user known to be of a particular age and race. These factors, in combination, may increase the probability of acute risk and prompt intervention or notification to the user 100, the caregiver 108, and/or a healthcare provider. For example, the wellness and healthcare monitoring device 102 may detect slowed or slurred speech, increased response latency to conversational partners, or increased syllable durations in speech productions, thus prompting notification or documentation of the biomarkers 104 in combination or alone. In this example, speech production and language behavior may be assessed in combination with pharmaceutical intake, pharmacotherapeutic interventions, or other medications to give feedback or information to the user 100 and/or those designated such as caregivers or researchers.

In an embodiment, the biomarkers 104 may include vocal biomarkers, physical biomarkers, environmental biomarkers, and/or audio biomarkers. In an embodiment, the vocal biomarkers may be associated with vocalizations (e.g., sound, communications, etc.) produced by the user 100. In an embodiment, the vocalizations may be associated with any sounds generated or emanating from the user 100, such as pain (e.g., groaning), distress (e.g., crying), social (e.g., conversation, rate, loudness, etc.), stomach grumbling, and so forth. In an embodiment, the vocal biomarkers may be determined based at least in part on characteristic(s) of the audio, such as tone, prosody, duration, amplitude, etc. In an embodiment, the physical biomarkers may be associated with physical activity of the user 100, such as walking, vitals, etc. The environmental biomarkers may be associated with the weather, a location of the user 100, temperature, humidity, etc. The audio biomarkers may be associated with a physiological, anatomical, behavioral, or mental state of the user 100. Other biomarkers, such as individual or situational biomarkers may be used to characterize experiences, activities, behaviors, actions, of the user 100. For example, the wellness and healthcare device 102 may classify a reduction in communicative contributions of the user 100 in noisy environments, during specific times of the day, or with specific interlocutors or companions. This complex behavioral data may contribute the ability to bear on meaningful quality of life, wellness, well-being interventions, suggestions, logging, or analyses. In the preceding example, a notification reminder may be produced to the user 100 to increase the frequency and amplitude of speech production and language contributions when a specific companion is detected by the wellness and healthcare device 102. This alert/notification could be produced in real-time and in retrospective fashion for later, off-line analysis.

In an embodiment, the welfare of the user 100 may be indicated by their behavior, activity, and interactions. The physical and mental health of the user 100 may be measured as a function of the behavior of the user 100, both inter- and intra-individually. Using predictors as mentioned herein, objective indicators are collected via sensor(s) 114 of the wellness and healthcare monitoring device 102, used in the classification/discrimination models, and interpreted as actionable information for the user 100 directly or for the caregiver 108.

In an embodiment, the biomarkers 104 may be determined based at least in part on the audio data 110 generated by microphone(s) 116. The audio data 110 includes but is not limited to spectral and temporal analyses of the sampled signal including fundamental frequency, harmonics of vocal tract including formant frequencies, respiratory support, amplitude characteristics, source-filter characteristics of vocal fold and laryngeal activity coupled to upper airway morphophysiology and anatomy, and vocal amplitude. These features are characterized and interpreted in combination, to infer physiological details of the user 100 (e.g., vocal tract length, oral aperture, laryngeal port motor control, lingual shape characteristics, etc.), physio-temporal details (e.g., fine- and gross-motor control, etc.), and range/tendency/error/entropy/stability in the observed distributions in the signal. Additionally, or alternatively, sensor data 118 generated by the sensor(s) 114 may be processed to determine the biomarkers 104. For example, sound captured by the microphone(s) 116 and sensor data 118 indicative of inhaling, exhaling, vocalizing (e.g., via vocal cord vibration, for example) generated by the sensor(s) 114 may be processed to determine the biomarkers 104.

The wellness and healthcare monitoring device 102 is shown including processor(s) 120 and memory 122, where the processor(s) 120 may perform various functions and operations associated with detecting the welfare, behavior, and/or pathological states 106, and the memory 122 may store instructions executable by the processor(s) 120 to perform the operations described herein. The wellness and healthcare monitoring device 102 may include any number of the sensor(s) 114 for detecting the vocalizations, behavior, etc. of the user 100 to determine the welfare, behavior, and/or pathological states 106.

In an embodiment, the microphone(s) 116 may include at least two microphones. Any number of microphone(s) 116, however, may be included within the wellness and healthcare monitoring device 102. The microphone(s) 116, in addition to capturing sound associated with the user 100, may be used for determining a directionality (e.g., time of flight, time-delay analytics, etc.) of sound and/or whether the sound corresponds to the user 100 and/or other sources (e.g., background, weather, etc.). The microphone(s) 116 may generate respective audio signals that are representative of sound within the environment. In an embodiment, the microphone(s) 116 may be spaced apart by a different distance from a mouth of the user 100, for example, to discern sounds that are generated by the user 100 from other sources of sound within the environment.

The wellness and healthcare monitoring device 102 may include speech-processing component(s) 124, such as speech recognition, digital signal processing (DSP), automatic speech processing and recognition (ASP and ASR), natural language processing (NLP), echo cancellation, noise reduction, beamforming, and the like to enable processing of vocalizations of the user 100. In an embodiment, the speech-processing component(s) 124 may include a multi-dimensional non-linear beamforming module used to process audio signals captured by the microphone(s) 116. Beamforming or spatial (multiplex) filtering is a signal processing technique for directional signal reception. Signals generated by the microphone(s) 116 may be processed in such a way that signals at particular angles experience constructive interference while others experience destructive interference. The beamforming techniques form multiple directional signals corresponding to different directions or orientations within the environment associated with the vocalizations. As sounds are received from a particular direction, the directional signal (e.g., formed beam) associated with that direction tends to exhibit more energy or signal strength than the other signals (or beams).

In an embodiment, the speech-processing component(s) 124 may be configured to perform speech recognition, such as automatic speech recognition (ASR), natural language processing (NLP), artificial neural networks (ANNs) such as large language models (LLMs), and/or natural language understanding (NLU) on the audio data 110 to identify words or phrases associated with the user speech, or may be configured to provide the audio data 110 to another device for performing the ASR, NLU, and/or NLP on the audio data 110. The speech-processing component(s) 124 may also reduce noise, identify same voice, double-talk, speaker segregation, talker in noise, differentiation in the cocktail party effect, echo, and/or attenuate any signal components associated with noise other than the user 100. In turn, after processing the audio signals, a clean-high quality audio signal (or the audio data 110) may be generated that is associated with the talker or the user 100.

The speech-processing component(s) 124 may also include a VAD component 126 that detects speech of the user 100, as compared to other sounds within the environment (e.g., background noise, speech from other persons in the environment, etc.) and/or other talkers. For example, the VAD component 126 may confirm that the sounds generated within the environment correspond to vocalizations of the user 100. The VAD component 126 may perform VAD techniques on the audio data 110 utilizing one or more VAD algorithms based on channel energy with long and short-term energies, sub-band long and short-term energies with combination logic, deep neural network (DNN) based VADs, or any other type of VAD algorithms, with hangover windows, to determine whether the audio data 110 is representative of vocalizations of the user 100. DNN approaches including multilayer perceptrons (MLPs), recurrent neural networks (RNNs), convolutional neural networks (CNNs), and post processing including hysteretic/duration-based thresholding, noise cancellation, or complex extension filtering may be employed.

More particularly, in an embodiment, the VAD component 126 may employ binary labeling, e.g., with speech and non-speech frames labeled 1 and 0, respectively. Using multiple microphone(s), VAD can be applied to each channel independently. Subsequently, each outputted binary label can be unified with a frame-by-frame logical sum operation. TDOA estimation is done, for example, using a generalized correlation method to estimate time delays. Using VAD in combination with TDOA in the wellness and healthcare monitoring device 102 having multiple microphone(s) 116, as described, readily distinguishes when the user 100 is speaking or not speaking. Calculating signal-to-noise ratio (SNR) may be based on a postprocessing algorithm such as hysteretic thresholding applied after the VAD is determined to discern which parts of detected sounds are of the user 100 and which parts are environmental noise or other talkers.

In an embodiment, other examples of the sensor(s) 114 of the wellness and healthcare monitoring device 102, such as piezoelectric sensors, accelerometers, etc., may capture signals that are processed to generate sensor data 118 that is used to determine the biomarkers 104. The sensor(s) 114 may additionally or alternatively include piezoelectric sensor(s), accelerometer(s), camera(s), temperature sensor(s), heart rate sensor(s), humidity sensor(s), inertial measurement unit (IMU) sensor(s), ECC sensor(s), gyroscope, blood glucose sensor(s), breathing sensor(s), GPS/RFID sensor(s), blood oxygen sensor(s), Hall sensor(s), passive infrared (PIR) sensor(s), etc. In an embodiment, the sensor(s) 114 may be used to identify vitals associated with the user 100, activities of the user 100, and non-activities of the user 100, as well as to characterize the vitals, activities, and/or non-activity. For example, the sensor(s) 114 may be used to determine activities of the user 100 (e.g., vocalizations, movement, eating, sleeping, etc.) and/or non-activates of the user 100 (e.g., non-vocalizations, breathing, heart rate, blood oxygen level, etc.). Moreover, the sensor(s) may be used to determine any number of sounds generated by or emanating from the user 100, whether audio, vocals, speech, murmuring, mumbling, etc.

The wellness and healthcare monitoring device 102 may detect vocal pathological or distress biomarkers, for example, by detecting sonic or other detectable human vocal biomarkers associated with a targeted disease condition or welfare disorder. In an embodiment, the wellness and healthcare monitoring device 102 detects one or more sonic markers associated with disease or distress, characterized by a diagnostic vocal, non-vocal acoustic, or non-acoustic detected signal.

The sensor data 118 generated from the sensor(s) 114, such as an IMU, may be used to determine vocalizations of the user 100, for example, based on experienced vibrations, accelerations, inhaling/exhaling, pressure changes, and so forth. The sensor(s) 114 may be calibrated to optimize function and increase processing fidelity. For example, in an embodiment, the wellness and healthcare monitoring device 102 may self-adjust or calibrate (in real-time, or throughout repeated use sessions). Calibration may yield more sensitive or situationally adjusted results when determining the welfare, behavior, and/or pathological states 106. The calibration and sensitization may operate by combining, for example, detection and coordinate processing of amplitude for the user 100 to refine or otherwise modify subsequent detection of the welfare, behavior, and/or pathological states 106.

The sensor data 118 collected by the sensor(s) 114 may be used in combination with the audio data 110. Timestamps associated with the audio data 110 and the sensor data 118 may be associated with one another to correlate the sensor data 118 with the audio data 110. In an embodiment, to effectively detect vocalization, the sensor(s) 114 be positioned in proximity to the clavicle, thyroid, or cricoid area (or equivalent), or near vocal production mechanism of the user 100 to better detect movement or pressure changes associated with vocalization, physiological state or change, and/or respiration.

The audio data 110 and/or sensor data 118 may be analyzed to determine the biomarkers 104. In an embodiment, the biomarkers 104 may be associated with the vocalizations of the user 100, such as pitch, intonation, tone, pauses, phonation, energy changes in the harmonic spectrum, fundamental frequency, voicing, windowed and absolute sonorant-peak rates, SNR, temporal and spectral voice characteristics, frequency, spectral/cepstral cues, vocal quality/stability (e.g., shimmer, jitter, harmonic to noise ratio), prosody, temporal output, and amplitude stability, and/or motor processes. In an embodiment, the biomarkers 104 may be characterized by duration, frequency, decibel level, amplitude, energy, etc. Biomarker(s) may also be associated with non-acoustic characteristic(s) of the user, such as motor processes, vitals, EEG, EKG, microsiemens, motion, fatigue, physical activity, posture, gait, location, as well as environment biomarkers such as temperature, air quality, location, date/time, etc. The biomarkers 104, as discussed above, may also be associated with movements of the user 100 (e.g., sleeping, running, walking, etc.), activities of the user (e.g., coughing, drinking, eating, etc.), the environment (e.g., weather, temperature, etc.).

In an embodiment, the wellness and healthcare monitoring device 102 may process the audio data 110 and/or the sensor data 118 to determine the biomarkers 104 for detecting the welfare, behavior, and/or pathological states 106, such as diseases, distresses, injuries, etc. The vocalizations of the user 100 may be used to deduce the welfare, behavior, and/or pathological states 106, or stated alternatively, the volitional or vegetative vocalizations of the user 100 may be compared against characteristics indicative of the welfare, behavior, and/or pathological states 106. In addition, the activities or non-activities of the user 100 may be used to determine the welfare, behavior, and/or pathological states 106, or whether the behavior of the user 100 is indicative of the welfare, behavior, and/or pathological states 106.

The reference biomarker(s), whether acoustic or non-acoustic, may be collected, sampled, generated, and/or stored as sample data, and analyzed in conjunction with audio data 110 and/or sensor data 118 detected by the wellness and healthcare monitoring device 102 to detect, diagnose, and manage a wide range of welfare, behavioral, and/or pathological states 106. Example diseases may include, but are not limited to, infectious diseases, metabolic disorders, gate disorders, respiratory diseases, gastrointestinal diseases, non-pathological conditions or states, sleeping, agitation, running, walking, psychological states, posture, etc. In an embodiment, the welfare of the user 100 may be associated with healthy, sick, diseased, etc., in discrete, graded, categorical, continuous, or probabilistic fashion. In an embodiment, the welfare of the user 100 may be used to classify or determine the behavioral and/or pathological states of the user 100. Pathological states may be related to, but are not limited to, respiration, feeding, water intake, movement, digestive, gastrointestinal, disease, metabolism, gate, and so forth. These pathological states may be correspondingly utilized to identify, characterize, diagnose, treat, etc. diseases (e.g., states, factors, etc.), disabilities, distresses, welfare disturbances, pathologies, activities, congenital defects, effects of medications, environmental effects, accidents, harmful physical events, etc. associated with the user 100. Input, sensor, and device data may also be used to categorize, classify, and otherwise use data from sources adjacent to the user 100 or interacting with the user 100.

In an embodiment, the biomarkers 104 may be compared to one or more predetermined thresholds or reference characteristic(s) indicative of the welfare, behavior, and/or pathological states 106, for example, to determine whether the behavior of the user 100 is indicative of a particular behavior or pathological state. In this manner, the biomarkers 104 may be compared to reference characteristics of user 100 with particular welfare, behavior, and/or pathological states 106 and user 100 without the welfare, behavior, and/or pathological states 106. Through this comparison, a statistical likelihood may be determined whether the vocalizations and/or behavior of the user 100 is indicative of the welfare, behavior, and/or pathological states 106.

For example, the wellness and healthcare monitoring device 102 may detect one or more biomarkers associated with congenital heart disease, characterized by heart murmurs, rapid breathing, shortness of breath, fast heartbeat, etc. (e.g., congenital heart disease biomarkers). Here, the wellness and healthcare monitoring device 102 may determine characteristic(s) 112 of the user 100 for determining the biomarkers 104 and, in turn, may compare the biomarkers 104 to reference biomarkers indicative of congenital heart disease. Based on this comparison, the wellness and healthcare monitoring device 102 may determine whether the characteristic(s) 112 of the user 100 indicate congenital heart disease. However, the wellness and healthcare monitoring device 102 may be helpful to detect any number of types of pathological conditions, distress states, and a wide range of other activities and circumstances detectable from a user 100 and/or their environment. Depending upon the welfare, behavioral, and/or pathological states 106, comparable sets of reference biomarkers may be employed.

In an embodiment, the wellness and healthcare monitoring device 102 may be configured to detect particular welfare, behavioral, and/or pathological states 106 of the user 100. In this sense, the wellness and healthcare monitoring device 102 may be tailored to the user 100 (e.g., with a known disease, for example) or may be used to diagnose welfare, behavioral, and/or pathological states 106. For example, the wellness and healthcare monitoring device 102 may be specifically programmed and/or optimized for the specific biomarkers 104 and/or characteristic(s) 112 that are already established or known for a behavioral and/or pathological state for a given disease, as well as the disease, the user 100, and/or the environments. For example, continuing with the above example, if a user 100 has congenital heart disease, the wellness and healthcare monitoring device 102 may be programmed to monitor for characteristics (e.g., heart rate, breathing patterns, movement, etc.) indicative of congenital heart disease, as compared to other diseases. Moreover, in an embodiment, the wellness and healthcare monitoring device 102, the microphone(s) 116, and/or the sensor(s) 114 may be calibrated to each disease, behavioral and/or pathological state of the disease, the user, the environment, location, etc. The comparison between the biomarker(s) 104 and the reference biomarkers or thresholds may determine a statistical likelihood of the welfare, behavioral, and/or pathological state of the user.

In an embodiment, the wellness and healthcare monitoring device 102 may determine fingerprint(s) 128 (or phenotype). The use of the fingerprint(s) 128 may increase an accuracy in detecting vocalizations of the user 100, discerning a type of vocalizations of the user 100, or discerning other sounds emanating within the environment. Moreover, the fingerprint(s) may be used to more accurately detect the biomarkers 104, and/or the welfare, behavior, and/or pathological states 106. For example, in an embodiment, the biomarkers 104 may be used to generate fingerprint(s) 128 (e.g., signature(s), audio signatures, acoustic fingerprint, etc.) associated with the user 100, and the fingerprint(s) 128 may be analyzed to determine whether the fingerprint(s) 128 is associated with, or includes, characteristics of the welfare, behavior, and/or pathological states 106. In an embodiment, the fingerprint(s) 128 may be compared to one or more reference fingerprints to detect the welfare, behavior, and/or pathological states 106. Capturing sounds of the user 100 allows for determining whether the user 100 includes a pathological state that warrants care, treatment, intervention, alert, etc. Each of the fingerprint(s) 128 may indicate biomarkers 104, and if a similarity between the audio fingerprint(s) 128 and a stored reference audio voiceprint exists, the welfare, behavior, and/or pathological states 106 may be detected. The fingerprint(s) 128 may be compared to any reference fingerprint(s) to determine whether any number of welfare, behavior, and/or pathological states 106 exist.

In an embodiment, the wellness and healthcare monitoring device 102 may output notification(s) 130 based at least in part on the welfare, behavior, and/or pathological states 106. The notification(s) 130 may provide feedback to the user 100, healthcare provider, and/or caregiver 108, for example, such that the caregiver 108 may administer aid, assist, treat, etc. the welfare, behavior, and/or pathological state 106. For example, if the user 100 has a disease, the caregiver 108 may administer medicine or provide other interventions. The notification(s) 130 may be output in real-time, or substantially real-time, to provide near-instantaneous feedback to the caregiver 108. In an embodiment, the notification(s) 130 may be associated with the biomarkers 104 that are detected. This may permit the caregiver 108 to make informed decisions about the care to administer, or whether to not to administer care. In an embodiment, the notification(s) 130 may be output on a mobile device 132 of the caregiver 108 and/or at the wellness and healthcare monitoring device 102.

In an embodiment, the notification(s) 130 may be audible, visual, haptic, and/or a combination thereof. The wellness and healthcare monitoring device 102 may include I/O component(s) 134 that output the notification(s) 130. For example, lighting elements (e.g., LEDs) may output light at different brightness levels, flashing at different durations, frequencies and/or specific sequences (e.g., numeric sequences of long and short flashes) in different modes, and/or by lighting in different colors. Speaker(s) may output an audible signal, such as a continuous prompt tone, sounding at different pitches or loudness in different modes, sounding at different durations, frequencies and/or specific sequences (e.g., numeric sequences of loud and soft, or high and low pitch sounds) in different modes. Other output components of the wellness and healthcare monitoring device 102 may include displays, touchscreens, etc. Example input devices may include button(s), switches, toggles, etc.

In an embodiment, the wellness and healthcare monitoring device 102 may store or have access to machine-learned (ML) model(s) 136. The ML model(s) 136 may be trained to analyze the audio data 110 and/or the sensor data 118 to determine the welfare, behavior, and/or pathological states 106 of the user 100. The ML model(s) 136 may determine the biomarkers 104 for determining the welfare, behavior, and/or pathological states 106. In an embodiment, the ML model(s) 136 may determine or generate metrics or score(s) associated with the audio data 110 and/or the sensor data 118 to determine whether the audio data 110 and/or the sensor data 118 are indicative of particular welfare, behavior, and/or pathological states 106. In an embodiment, the score(s) may relate to a probability or likelihood that the vocalizations, activity, vitals, behavior, etc. of the user 100 is associated with certain welfare, behavior, and/or pathological states 106. In other words, the score(s) output by the ML model(s) 136 may be machine-learned scores.

Machine learning generally involves processing a set of examples (called “training data”) to train a machine learning model(s). Once trained, a machine learning model(s) is a learned mechanism that can receive new data as input and estimate or predict a result as output. For example, a trained machine learning model may comprise a classifier that is tasked with classifying unknown input (e.g., an unknown audio or sensor data) as one of multiple class labels. In some cases, a trained machine learning model is configured to implement a multi-label classification task. Additionally, or alternatively, a trained machine learning model including, for example, Bayesian machine models (BMMs) relying on Bayesian networks or Dirichlet processes in complex hyperparameter environments or dynamical systems complex networks may be trained to infer a probability or a set of probabilities, for a classification task based on unknown data received as input. In the context of the present disclosure, the unknown input may be the audio data 110 and/or the sensor data 118 that is associated with the user 100, and the ML model(s) 136 may be tasked with outputting the score that indicates or otherwise relates to, a probability or residual probability of the user 100 having a particular welfare, behavior, and/or pathological state 106. Additionally, the scores may indicate or otherwise relate to a probability of the biomarkers 104 for determining whether the vocalizations, activities, vitals, and/or behavior of the user 100 is associated with the welfare, behavior, and/or pathological states 106. Other data, such as medications of the user 100, a history of the user 100, etc. may be provided as an input to the ML model(s) 136. Such information, for example, may be stored within a profile of the user 100 and others who have contributed data to the sensor(s) on the wellness and healthcare monitoring device 102.

The training data used to train ML model(s) 136 may include various data types. In general, training data for machine learning may include two or more components, features and labels. However, in an embodiment, the training data used to train the ML model(s) 136 may be unlabeled, sparsely categorized, or underspecified. Accordingly, the ML model(s) 136 may be trainable using any suitable learning technique, such as supervised learning, unsupervised learning, semi-supervised learning, reinforcement learning, and so on. The features included in the training data may be represented by a complex set of features, such as in the form of an n-dimensional feature vector of quantifiable information about an attribute of the training data. The following is a list of example features that can be included in the training data for training the ML model(s) 136 described herein. However, it is to be appreciated that the following list of features is non-exhaustive, and features used in training may include additional features not described herein, and, in some cases, some, but not all, of the features listed herein. Example features included in the acoustic training data may include, without limitation, pitch, intonation, tone, pauses, articulation, decreased energy in the harmonic spectrum, fundamental frequency, SNR, temporal and spectral vocal characteristics, frequency, spectral/cepstral cues, vocal quality/stability, prosody, temporal output, and amplitude stability, and/or motor processes. Example features included in the behavioral training data may include, without limitation, movement, position, eye and pupil orientation and aperture, facial positioning, eating/drinking/walking/running/sleeping, passive/inactive states, personal hygiene and associated personal care, motion, relational activity, and/or volitional/vegetative fine and gross motor activity. Example features included in the environmental training data may include, without limitation, geolocation, relative location, interpersonal space, orientation in social time and/or space, psychosocial relationships, elevation, humidity, altitude, ambient temperature, particulate matter, air quality, ambient acoustic noise, heat, light, vibration, and/or olfactory influences. In an embodiment, the features included within the training data may be associated with vocalizations and/or behavior of the user 100 that contains welfare, behavior, and/or pathological states 106 and/or does not contain welfare, behavior, and/or pathological states 106.

In an embodiment, as part of the training process, weights may be applied to a set of features included in the training data, as derived from the historical data. In an embodiment, the weights that are set during the training process may apply to parameters that are internal to the ML model(s) 136 (e.g., weights for neurons in a hidden layer of a neural network). These internal parameters of the ML model(s) 136 may or may not map one-to-one with individual input features of the set of features. The weights may indicate the influence that any given feature, parameter, or characteristic has on the score that is output by the ML model(s) 136.

The ML model(s) 136 represent a single model or an ensemble of base-level machine learning models and may be implemented as any type of machine learning model. For example, suitable machine learning models for use with the techniques and systems described herein include, without limitation, neural networks, tree-based models, support vector machines (SVMs), kernel methods, random forests, splines (e.g., multivariate adaptive regression splines), hidden Markov model (HMMs), Kalman filters (or enhanced Kalman filters), Bayesian networks (or Bayesian belief networks), expectation maximization, genetic algorithms, linear regression algorithms, complex networks, nonlinear regression algorithms, logistic regression-based classification models, or an ensemble thereof. An “ensemble” can comprise a collection of machine learning models whose outputs (predictions) are combined, such as by using weighted averaging or voting. Models may be predictive or descriptive. The individual machine learning models of an ensemble can differ in their expertise, and the ensemble can operate as a committee of individual machine learning models that is collectively “smarter” than any individual machine learning model of the ensemble.

In an embodiment, and as noted above, the ML model(s) 136 may identify or determine the characteristic(s) 112, such as the biomarkers 104, for comparison against the reference characteristic(s). The ML model(s) 136 may learn to identify complex relationships between the biomarkers 104 to determine whether the vocalizations, activity, vitals, and/or behavior are associated with the welfare, behavior, and/or pathological states 106. For example, the ML model(s) 136 may learn to associate certain biomarkers 104 with whether the user has, or does not have, a certain welfare, behavior, and/or pathological state 106.

In an embodiment, the ML model(s) 136 may learn to predict that the vocalizations and/or behavior is indicative of a welfare, behavior, and/or pathological state 106 by attributing corresponding score(s). In this manner, biomarkers 104, for example, with low scores (e.g., below threshold) may not include a particular welfare, behavior, and/or pathological state 106, while the biomarkers 104 with high scores (e.g., above threshold) may include a particular welfare, behavior, and/or pathological state 106. Although the use of a threshold is described as one example way of providing labeling (i.e., replaced or maintained), other techniques are contemplated, such as clustering algorithms, or other statistical approaches that use the trust scores may be used. The ML model(s) 136 is/are retrainable with new data to adapt the ML model(s) 136.

In addition to processing the audio data 110 and/or the sensor data 118 to detect the welfare, behavior, and/or pathological states 106, the wellness and healthcare monitoring device 102 may utilize other data. For example, the wellness and healthcare monitoring device 102 may have access to data stored within a profile 138. The data may indicate demographics of the user 100, environmental factors, medications of the user 100, a history of medical diagnoses, condition, etc. of the user 100, and so forth. This data may additionally, or alternatively be used to treat, detect, manage, and/or prevent the welfare, behavior, and/or pathological states 106. For example, incorporating user-specific, treatment-related, non-vocal data may be used to more effectively treat and manage the welfare, behavior, and/or pathological states 106.

The biomarkers 104 may be stored in association with the profile 138. The profile 138 may also store the audio data 110 and/or the sensor data 118 in association with the user 100. The caregiver 108 may use such data to further refine therapeutic benefits made possible by the wellness and healthcare monitoring device 102. The audio data 110 and/or sensor data 118 may also be stored in the memory 122 of the wellness and healthcare monitoring device 102 and provided (e.g., downloaded) to other devices.

The wellness and healthcare monitoring device 102 may be communicatively coupled to one or more devices, such as the mobile device 132 and/or remote computing resource(s) 140 over network(s) 142. The network(s) 142 may include any viable communication technology, such as wired and/or wireless modalities and/or technologies. The network(s) 142 may include any combination of Personal Area Networks (PANs), Local Area Networks (LANs), Campus Area Networks (CANs), Metropolitan Area Networks (MANs), extranets, intranets, the Internet, short-range wireless communication networks (e.g., ZigBee, Bluetooth, etc.) Wide Area Networks (WANs)—both centralized and/or distributed—and/or any combination, permutation, and/or aggregation thereof. The wellness and healthcare monitoring device 102 includes suitable network interface(s) 144 for communicating over the network(s) 142.

In an embodiment, the wellness and healthcare monitoring device 102 may send the audio data 110 and/or the sensor data 118 to the remote computing resource(s) 140 for processing. The remote computing resource(s) 140 may receive the audio data 110 and/or the sensor data 118 from any number of the wellness and healthcare monitoring devices 102 disposed within the environment. The remote computing resource(s) 140 may have access to the components (e.g., ML model(s) 136) or additional components for processing the audio data 110 and/or the sensor data 118. In an embodiment, the remote computing resource(s) 140 may process the audio data 110 and/or the sensor data 118, determine the biomarkers 104, determine the welfare, behavior, and/or pathological states 106, and then communicate the notification(s) 130 to the wellness and healthcare monitoring device 102, the mobile device 132, or other devices. Any level of split processing may be performed by the wellness and healthcare monitoring device 102, the mobile device 132, the remote computing resource(s) 140, and/or other devices, systems, networks, etc.

Although the techniques are described herein as being performed by the wellness and healthcare monitoring device 102, the techniques may be embodied as software or an application operating on a device (e.g., mobile phone). Suitable APIs may interact and/or utilize sensor(s) of device(s), such as camera(s), microphone(s), location, temperature, etc. to carry out the operations described herein.

The remote computing resource(s) 140 may be implemented as one or more servers and may, in an embodiment, form a portion of a network-accessible computing platform implemented as a computing infrastructure of processors, storage, software, data access, etc. that is maintained and accessible via a network such as the Internet. The remote computing resource(s) 140 does not require end-user knowledge of the physical location and configuration of the system that delivers the services. Common expressions associated with the remote computing resource(s) 140 may include “on-demand computing”, “software as a service (SaaS)”, “platform computing”, “network-accessible platform”, “cloud services”, “data centers”, etc.

As used herein, a processor, such as the processor(s) 120 may include multiple processors and/or a processor having multiple cores. Further, the processor(s) 120 may comprise one or more cores of different types. For example, the processor(s) 120 may include application processor units, graphic processing units, and so forth. In one implementation, the processor(s) 120 may comprise a microcontroller and/or a microprocessor. The processor(s) 120 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 120 may possess its own local memory, which also may store program components, program data, and/or one or more operating systems.

Memory, such as the memory 122 may include volatile and nonvolatile memory, removable and non-removable media implemented in any method or technology for storage of information, such as computer-readable instructions, data structures, program component, or other data. Such memory may include but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory may be implemented as computer-readable storage media (“CRSM”), which may be any available physical media accessible by the processor(s) to execute instructions stored on the memory. In one basic implementation, CRSM may include random access memory (“RAM”) and Flash memory. In other implementations, CRSM may include but is not limited to, read-only memory (“ROM”), electrically erasable programmable read-only memory (“EEPROM”), or any other tangible medium that can be used to store the desired information and which can be accessed by the processor(s) 120. The memory 122 is an example of non-transitory computer-readable media. The memory 122 may store an operating system and one or more software applications, instructions, programs, and/or data to implement the methods described herein and the functions attributed to the various systems.

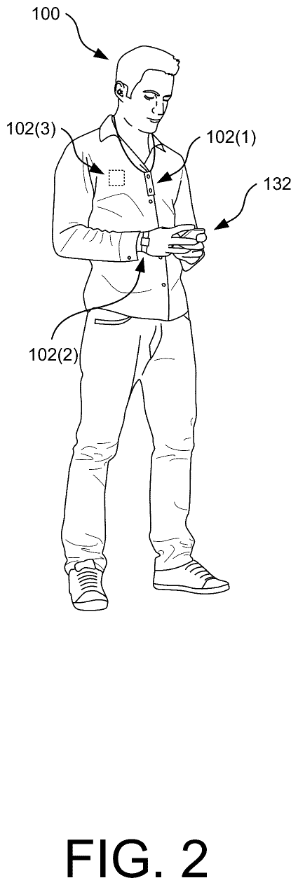

FIG. 2. illustrates a plurality of the wellness and healthcare monitoring devices 102 being worn by the user 100, according to examples of the present disclosure. As introduced above, the wellness and healthcare monitoring device 102 may take any shape, may be coupled or otherwise attached to the user 100 in different manners, and/or may have different functionalities.

For example, the user 100 may wear a first wellness and healthcare monitoring device 102(1), a second wellness and healthcare monitoring device 102(2), and a third healthcare monitoring device 102(3). The first wellness and healthcare monitoring device 102(1) may be worn around a neck of the user 100, for example, via a lanyard. The second wellness and healthcare monitoring device 102(2) may be worn around a wrist of the user, for example, as a watch. The third wellness and healthcare monitoring device 102(3) may be worn as a stick-on array, patch, etc. Each of the wellness and healthcare monitoring devices 102(1)-(3) may have suitable components to permit their operation, such as sensors, batteries, processor(s), etc., similar to those discussed above with regard to FIG. 1.

In an embodiment, the wellness and healthcare monitoring device(s) 102(1)-(3) may have different functionalities or different sensor(s) for determining the biomarkers 104 of the user 100. For example, the first wellness and healthcare monitoring device 102(1) may have microphone(s) to capture sounds generated by the user 100, the second wellness and healthcare monitoring device 102(2) may have a pulse oximeter sensor, a heart rate sensor, etc., and the third wellness and healthcare monitoring device may have a temperature sensor, accelerometer, etc. As such, the wellness and healthcare monitoring device(s) 102(1)-(3) may include different sensor(s) for generating the sensor data 118 for determining the biomarkers.

In an embodiment, the wellness and healthcare monitoring device(s) 102(1)-(3) may communicatively couple one another. Additionally, or alternatively, the wellness and healthcare monitoring device(s) 102(1)-(3) may communicatively couple to the mobile device 132 and/or the remote computing resource(s) 140. For example, the wellness and healthcare monitoring device(s) 102(1)-(3) may send sensor data 118 to the mobile device 132, whereby the mobile device 132 may analyze the sensor data 118 across the wellness and healthcare monitoring device(s) 102(1)-(3) to determine the biomarkers 104 and/or the welfare, behavioral, and/or pathological states 106.

Although the wellness and healthcare monitoring device(s) 102(1)-(3) are shown being worn by the user 100, the wellness and healthcare monitoring device(s) 102(1)-(3) may communicatively couple to other wellness and healthcare monitoring device(s), whether worn by the user 100 or not worn by the user 100 (e.g., stand-alone device).

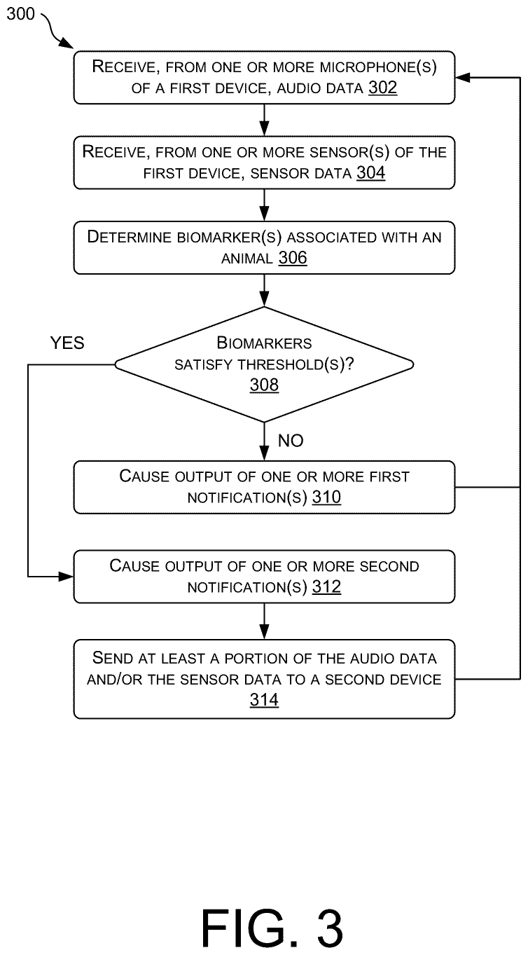

FIG. 3 illustrates an example process 300 (e.g., method) related to determining whether welfares, behavioral, and/or pathological states 106 of the user 100, according to examples of the present disclosure. The process 300 described herein is illustrated as collections of blocks in logical flow diagrams, which represent a sequence of operations or acts, some or all of which may be implemented in hardware, software, or a combination thereof. In the context of software, the blocks may represent computer-executable instructions stored on one or more computer-readable media that, when executed by one or more processors, program the processors to perform the recited operations or acts. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process 300 or alternative processes, and not all of the blocks need be executed. For discussion purposes, the process 300 is described with reference to the environments, devices, architectures, diagrams, and systems described in the examples herein, such as, for example those described with respect to FIGS. 1 and 2, although the process 300 may be implemented in a wide variety of other environments, architectures, and systems.

At 302, the process 300 may include receiving audio data from one or more microphone(s) of a first device. For example, the wellness and healthcare monitoring device 102 may receive the audio data 110 as generated from the microphone(s) 116. In an embodiment, the audio data 110 may be at least partially processed or unprocessed (e.g., filtered). In an embodiment, rather than receiving audio data 110, the wellness and healthcare monitoring device 102 may receive audio signals from the microphone(s) 116.

At 304, the process 300 may include receiving sensor data from one or more sensor(s) of the first device. For example, the wellness and healthcare monitoring device 102 may receive the sensor data 118 as generated from the sensor(s) 114. In an embodiment, the sensor data 118 may be at least partially processed or unprocessed (e.g., filtered). In an embodiment, rather than receiving sensor data 118, the wellness and healthcare monitoring device 102 may receive sensor signals from the sensor(s) 114. The sensor(s) 114 may include accelerometer(s), piezoelectric sensor(s), IMUs, GPS, etc. The wellness and healthcare monitoring device 102 may receive the sensor data 118 from any number of sensor(s) 114, and/or in some instances, the wellness and healthcare monitoring device 102 may receive sensor data 118 from other wellness and healthcare monitoring devices 102 that are communicatively coupled to the wellness and healthcare monitoring device 102. Any number of the sensor(s) 114 may be disposed across the wellness and healthcare monitoring devices 102 and/or the number and/or type of the sensor(s) 114 may be based at least in part on an application of the wellness and healthcare monitoring device 102, the user 100, and/or the welfare, behavioral, and/or pathological state 106 being examined, etc. For example, if the user 100 has a medical history of a certain disease, or the wellness and healthcare monitoring device 102 is being employed to treat, manage, etc. a certain disease, the types of sensor(s) 114 on the wellness and healthcare monitoring device 102 may be tailored accordingly.

At 306, the process 300 may include determining biomarker(s) associated with the user. In an embodiment, the biomarker(s) 104 may be associated with determining welfare, behavioral, and/or pathological states 106. Any number of the biomarkers 104 may be determined from the audio data 110 and/or the sensor data 118. The biomarkers 104 may be associated with sounds, vocalizations, activities, non-activities, vitals, etc. of the user 100, as well as characteristics of an environment of the user 100. As non-limiting examples, the biomarkers 104 may include a pitch of vocalizations of the user 100, intonations of vocalizations of user 100, tones associated with the vocalizations of user 100, changes associated with the vocalizations of user 100, weather within an environment of the user 100, movement of the user 100, eating/drinking/walking/sleeping/etc. habits associated with the user 100, frequency, spectral/cepstral cues, vocal quality/stability, prosody, temporal output, and amplitude stability, motor processes, and so on. In an embodiment, the biomarkers 104 may be acoustic-based or non-acoustic based. Moreover, in an embodiment, the biomarkers 104 may be based at least in part on the user 100 and/or the use of the wellness and healthcare monitoring device 102. For example, if the wellness and healthcare monitoring device 102 is being employed to treat, manage, etc. a certain disease, the wellness and healthcare monitoring device 102 may be employed to determine the presence, or absence, of certain biomarkers 104 for the disease.

In an embodiment, the ML model(s) 136 may be used to process the audio data 110 and/or the sensor data 118 for determining the biomarkers 104. For example, the ML model(s) 136 may be previously trained to determine the biomarkers 104. In an embodiment, the ML model(s) 136 may be selected from a plurality of ML model(s) 136 based at least in part on the user 100 (e.g., age, medical history, etc.) and/or the welfare, behavioral, and/or pathological states 106 the wellness and healthcare monitoring device 102 is being employed to monitor. In this sense, the wellness and healthcare monitoring device 102 may be tailored, programmed, etc. according to the specifics of the user 100 or caretaker/healthcare provider in order more accurately detect the presence, state, condition, etc. of certain diseases.

In an embodiment, the ML model(s) 136 may receive the audio data 110 and/or the sensor data 118 as an input, and output an indication associated with whether the audio data 110 and/or the sensor data 118 are indicative of the biomarkers 104. In an embodiment, the ML model(s) 136 may output a score associated with the biomarkers 104, where the score may indicate likelihood or probability of the audio data 110 and/or the sensor data 118 being indicative of the biomarkers 104. In an embodiment, the ML model(s) 136 may output a score associated with the welfare, behavioral, and/or pathological states 106 of the user 100. Moreover, in an embodiment, the ML model(s) 136 may determine the fingerprint(s) 128 for comparison to reference fingerprint(s)/phenotype(s) associated with the welfare, behavioral, and/or pathological states 106, for determining whether the vitals, behavior, etc. of the users 100 is indicative of the welfare, behavioral, and/or pathological states 106.

At 308, the process 300 may include determining whether the biomarker(s) satisfy predetermined threshold(s). For example, the biomarkers 104 may be compared to reference biomarker(s), or characteristic(s) that are indicative of the welfare, behavioral, and/or pathological states 106. Any number of the biomarkers 104 may be considered when determining whether the biomarkers 104 are indicative of the welfare, behavioral, and/or pathological states 106. In an embodiment, the ML model(s) 136 may be used to determine whether the biomarkers 104 are indicative of satisfying threshold(s) associated with the welfare, behavioral, and/or pathological states 106. In an embodiment, score(s) output by the ML model(s) 136 may be compared to a threshold score for determining whether the score satisfies certain thresholds associated with the welfare, behavioral, and/or pathological states 106. Moreover, as referenced above, in an embodiment, the fingerprint(s) 128 may be determined for comparison with one or more reference fingerprints to determine the welfare, behavioral, and/or pathological states 106 of the user 100. If, at 308, the process 300 determines that the biomarkers 104 do not satisfy the threshold(s), the process 300 may follow the “NO” route and proceed to 310.

At 310, the process 300 may include causing output of one or more first notification(s). For example, the I/O component(s) 134 of the wellness and healthcare monitoring device 102 may output notification(s) 130, where the notification(s) 130 may indicate that the audio data 110 and/or the sensor data 118 is not associated with a particular welfare, behavioral, and/or pathological state 106. In an embodiment, the notification(s) 130 may be output at the wellness and healthcare monitoring device 102, and/or the mobile device 132 of the caregiver 108. From 310, the process 300 may proceed to 302, whereby the process 300 may continue to receive the audio data 110 and/or the sensor data 118 for determining the welfare, behavioral, and/or pathological states 106.

Returning to 308, if the process 300 determines that the biomarker(s) 104 satisfy the threshold(s), the process 300 may follow the “YES” route and proceed to 312. At 312, the process 300 may include causing output of one or more second notification(s). For example, the I/O component(s) 134 of the wellness and healthcare monitoring device 102 may output notification(s) 130, where the notification(s) 130 may indicate the welfare, behavioral, and/or pathological states 106.

At 314, the process 300 may include sending at least a portion of the audio data and/or the sensor data to a second device. For example, the wellness and healthcare monitoring device 102 may send the audio data 110 and/or the sensor data 118 to the mobile device 132 and/or the remote computing resource(s) 140. In an embodiment, the mobile device 132 and/or the remote computing resource(s) 140 may further process the audio data 110 and/or the sensor data 118, may store/log the audio data 110 and/or the sensor data 118 to maintain a history of the user 100. The audio data 110 and/or the sensor data 118 may be used to by the mobile device 132 and/or the remote computing resource(s) 140 to retrain the ML model(s) 136, confirm the biomarker(s) 104 as determined by the wellness and healthcare monitoring device 102, and/or the welfare, behavioral, and/or pathological states 106 determined by the wellness and healthcare monitoring device 102. In addition, the mobile device 132 may output the notification(s) 130 indicative of the welfare, behavioral, and/or pathological states 106. From 314, the process 300 may loop to 302, whereby the wellness and healthcare monitoring device 102 may continue to process and analyze the audio data 110 and the sensor data 118 associated with the user 100.

While various examples and embodiments are described individually herein, the examples and embodiments may be combined, rearranged, and modified to arrive at other variations within the scope of this disclosure.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

What is claimed is:

1. A device to be worn by a user, the device comprising:
one or more microphones;
an output component;
a network interface;
one or more processors; and
one or more non-transitory computer-readable media storing computer-executable instructions that, when executed by the one or more processors, cause the device to perform acts comprising:
receiving, from the one or more microphones, audio data associated with sound captured within an environment of the user,
determining, based at least in part on the audio data, that the sound is associated with the user,
determining, based at least in part on the sound being associated with the user, one or more biomarkers associated with the sound,
determining a threshold associated with the user having a pathological or behavioral state, process, or change,
determining, based at least in part on the one or more biomarkers satisfying the threshold, that the sound is indicative of the user having the pathological or behavioral state, process, or change,
causing, based at least in part on the one or more biomarkers satisfying the threshold, output of a first notification on the output component, and
sending data associated with a second notification to be output on an electronic device associated with a caregiver of the user.

2. The device of claim 1, further comprising a sensor, the acts further comprising receiving, from the sensor, sensor data, wherein:
determining that the sound is associated with the user is based at least in part on the sensor data; and
determining the one or more biomarkers is based at least in part on the sensor data.

3. The device of claim 1, the acts further comprising:
receiving, from the one or more microphones, second audio data associated with a second sound captured within the environment;
determining, based at least in part on the second audio data, that the second sound is associated with the environment; and
determining one or more second biomarkers associated with the second sound,
wherein determining that the sound is indicative of the user having the pathological or behavioral state is based at least in part on the one or more second biomarkers.

4. The device of claim 1, the acts further comprising:
determining a signature associated with the pathological or behavioral state;
determining, based at least in part on the one or more biomarkers, an audio signature associated with the sound; and
determining a similarity between the audio signature associated with the pathological or behavioral state and the audio signature associated with the sound,
wherein determining that the sound is indicative of the user having the pathological or behavioral state is based at least in part on the similarity.

5. The device of claim 1, further comprising one or more attachment mechanisms for coupling the device to the user.

6. The device of claim 1, wherein determining the one or more biomarkers is based at least in part on:
providing, as an input to a machine-learned (ML) model trained to identify the pathological or behavioral state, the audio data; and
receiving, as an output from the ML model, an indication associated with the one or more biomarkers.

7. The device of claim 1, wherein the one or more biomarkers include at least one of a pitch of the sound, a tone associated with the sound, a pause in the sound, a duration of the sound, or an amplitude of the sound.

8. A device comprising:
one or more sensors;
one or more output components;
one or more processors; and
one or more non-transitory computer-readable media storing computer-executable instructions that, when executed by the one or more processors, cause the device to perform acts comprising:
receiving, from the one or more sensors, data,
determining that the data is indicative of sound generated from a user,
determining, based at least in part on the data being indicative the sound generated from the user, one or more biomarkers associated with the sound, the one or more biomarkers including at least an amplitude associated with the sound,
determining that the one or more biomarkers fail to satisfy a threshold associated with a pathological or behavioral state or change of the user, and
causing, based at least in part on the one or more biomarkers failing to satisfy the threshold, output of a notification via the one or more output components.

9. The device of claim 8, wherein the one or more sensors comprise at least one of:
a microphone; or
at least one of an internal measurement unit (IMU), an accelerometer, a gyroscope, or a piezoelectric sensor.

10. The device of claim 8, wherein the one or more output components comprise at least one of a lighting element or a speaker.

11. The device of claim 8, the acts further comprising sending a second notification to an electronic device.

12. The device of claim 8, wherein the device is configured to be worn by the user.

13. The device of claim 8, the acts further comprising:
receiving, from the one or more sensors, second data;
determining that the second data is indicative of second sound generated from the user;
determining, based at least in part on the second data being indicative the second sound generated from the user, one or more second biomarkers associated with the second sound;
determining that the one or more second biomarkers satisfy the threshold, and
causing, based at least in part on the one or more second biomarkers satisfying the threshold, output of a third notification via the one or more output components.

14. The device of claim 8, wherein determining the one or more biomarkers is based at least in part on:
providing, as an input to a machine-learned (ML) model trained to identify the pathological or behavioral state; and
receiving, as an output from the ML model, an indication associated with the one or more biomarkers.

15. The device of claim 8, wherein the one or more sensors comprise at least one of a microphone, an accelerometer, an inertial measurement unit (IMU), a GPS, or a temperature sensor.

16. A device comprising:
a first microphone;
a second microphone;
one or more output components;
one or more processors; and
one or more non-transitory computer-readable media storing computer-executable instructions that, when executed by the one or more processors, cause the device to perform acts comprising:
receiving, from the first microphone, first audio data associated with a sound captured in an environment,
receiving, from the second microphone, second audio data associated with the sound,
determining, based at least in part on the first audio data and the second audio data, that the sound is associated with a user,
determining, based at least in part on the sound being associated with the user, one or more characteristics associated with the sound,
determining, based at least in part on the one or more characteristics, that the sound is indicative of a pathological or behavioral state of the user, and
causing, based at least in part on the one or more characteristics being indicative of the pathological or behavioral state, output of a notification via the one or more output components.

17. The device of claim 16, the acts further comprising sending data associated with the one or more characteristics to an electronic device.

18. The device of claim 16, further comprising one or more sensors that include at least one of an accelerometer, a gyroscope, an internal measurement unit (IMU), or a piezoelectric sensor, the acts further comprising receiving, from the one or more sensors, sensor data, wherein:
determining that the sound is associated with the user is based at least in part on the sensor data; and
determining the one or more characteristics associated with the sound is based at least in part on the sensor data.

19. The device of claim 16, the acts further comprising determining one or more characteristics associated with an environment of the user, and wherein determining that the sound is indicative of the pathological or behavioral state is based at least in part on the one or more characteristics associated with the environment.

20. The device of claim 16, wherein determining the one or more characteristics is based at least in part on:
providing, as an input to a machine-learned (ML) model trained to identify the pathological or behavioral state; and
receiving, as an output from the ML model, an indication associated with the one or more characteristics.
